(19) United States
(12) Patent Application Publication
(10) Pub. No.: US 2010/0137752 A1
(43) Pub. Date: Jun. 3, 2010

(54) Medical Apparatus For Treatment Of The Human Or Animal Body By Mechanical Pressure Waves Or Shock Waves

(76) Inventors: Gerold Heine, Ohningen (DE); Klaus-Martin Irion, Emmingen-Liptingen (DE); Ernst H. Marlinghaus, Bottighofen (CH); Manfred Schulz, Taegerwilen (CH); Pavel Novak, Stetten (CH)

Correspondence Address: ST. ONGE STEWARD JOHNSTON & REENS, LLC, 986 BEDFORD STREET, STAMFORD, CT, 06905-5619, US

(21) Appl. No.: 12/627,745

(22) Filed: Nov. 30, 2009

Related U.S. Application Data
(63) Continuation of application No. PCT/EP2008/003883, filed on May 15, 2008.

(30) Foreign Application Priority Data
May 31, 2007 (DE) 20 2007 007 920.6

(51) Int. Cl. A61N 7/00; A61B 18/14; A61B 18/20

(52) U.S. Cl. 601/2; 606/41; 606/13

(57) ABSTRACT

A medical apparatus for treatment of the human or animal body by mechanical pressure waves or shock waves, comprises a device designed to generate the pressure waves or shock waves. The device for generating the pressure waves or shock waves have at least one applicator part which can be subjected to impacts at a proximal end in order to generate the pressure waves or shock waves and, at a distal end, applies the pressure waves or shock waves into the body. The apparatus is additionally designed to generate outside the body, and to apply into the body, at least one further physiological action parameter chosen from the group consisting of electrical current, electro-magnetic waves, in particular light, ultrasound and/or a further mechanical action.

### CROSS REFERENCE TO RELATED APPLICATIONS

The present application is a continuation of pending international patent application PCT/EP2008/003883 filed on May 15, 2008 which designates the United States and which claims priority of German utility model application no. 20 2007 007 920.6 filed on May 31, 2007.

### BACKGROUND OF THE INVENTION

The invention generally relates to medical apparatus for treatment of the human or animal body by mechanical pressure waves or shock waves. Most specifically, the invention relates to medical apparatus for treatment of the human or animal body by mechanical pressure waves or shock waves of the type comprising a device designed to generate the pressure waves or shock waves and having at least one applicator part which can be subjected to impacts at a proximal end in order to generate the pressure waves or shock waves and which, at a distal end, applies the pressure waves or shock waves into the body.

Within the meaning of the present invention, “treatment of the human or animal body” signifies, for example, treatment of soft tissue, in particular for alleviating pain, treatment of bone tissue, crashing of stones in the body, removal of plaque from vessels, treatment of teeth, but also removal of bone cement or driving-in of bone nails or wires.

A medical apparatus known from document DE 197 25 477 C2 is used for treatment of bone fractures, enthesopathies, tendinopathies and also periodontosis. Another field of use of this known apparatus is the treatment of pain in the soft-tissue areas of the locomotor and support system located near bone. All these uses of the known medical apparatus can also be carried out with the medical apparatus according to the present invention.

The known apparatus comprises, as its device for generating the pressure waves or shock waves, an applicator part which is subjected to impacts by a periodically movable percussion part. The percussion part is for this purpose moved to and from along an acceleration path, said percussion part being driven by compressed air between a proximal starting position and a distal end position, in which the percussion part impacts against the applicator part.

After they have been applied into the body, the pressure waves or shock waves generated by the applicator part deploy a mechanical pressure wave or shock wave effect in the area of the body to be treated.

### SUMMARY OF THE INVENTION

It is an object of the present invention to improve an apparatus of the type mentioned at the outset in such a way that the action of the pressure waves or shock waves in the human body, in particular the therapeutic action on tissue, is further improved.

According to a first aspect of the invention, a medical apparatus for treatment of the human or animal body by mechanical pressure waves or shock waves is provided, comprising a device designed to generate the pressure waves or shock waves, the device having at least one applicator part having a proximal end and a distal end, and which can be subjected to impacts at the proximal end in order to generate the pressure waves or shock waves and which applies the pressure waves or shock waves into the body at the distal end, wherein the apparatus is additionally designed to generate outside the body, and to apply into the body, at least one further physiological action parameter chosen from the group consisting of electrical current, electro-magnetic waves, ultrasound, a further mechanical action beyond the pressure waves or shock waves.

The apparatus according to the invention thus differs from the known apparatus in that it is not only the mechanical action of the pressure waves or shock waves that is deployed in the area of the body to be treated, but also at least one additional action based on current, light and/or ultrasound. It is also possible for another mechanical action parameter beyond the pressure waves and shock waves to be generated and applied into the body, as will be described below. The invention is based on the recognition that the action of the mechanical pressure waves or shock waves can be improved if the area of the body to be treated, for example soft tissue, is acted upon by means of current, light and/or ultrasound, which increases the therapeutic action of the mechanical pressure waves or shock waves by, for example, increasing the circulation of blood and stimulating cells in the tissue that is to be treated.

In preferred embodiments, the at least one other action parameter is generated by the applicator part itself, by a cap on the distal side of the applicator part and/or by an applicator-retaining part fixed to the housing, and is applied thereby into the body.

For this purpose, in practical embodiments, provision is preferably made for the applicator part to comprise at least one electrode that can be supplied with current, this also including the case where the applicator part itself serves as electrode, where the applicator part has a cap arranged on the distal side and having at least one electrode that can be supplied with current, and/or where the at least one electrode is arranged in or on a headpiece of the housing.

The electrode is preferably circular in order to provide a large surface for application of the current, which is preferably a therapeutic alternating current, and therefore to obtain a large surface for action on the area of the body to be treated. The current can flow back capacitively without counter-electrode, via a neutral electrode on the patient's body or via a counter-electrode on the apparatus, for example on the applicator part or on the housing.

In another preferred embodiment, the apparatus is equipped with a power supply source or is connected to such a power supply source, which supplies the at least one electrode with high frequency current.

This embodiment is in particular advantageous for the treatment of cellulite or skin folds with the apparatus according to the invention, because collagen fibers can be heated with the high frequency current to such an extent, that the collagen fibers shrink. Bipolar high frequency current is preferably used as high frequency current, if the skin is to be heated superficially to this purpose, until the shrinking of the collagen fibers occurs, and/or monopolar high frequency current is used, which achieves a higher depth effect and, thus, heats the tissue in the depth to an extent until there occurs a shrinking of collagen fibers. The combination of the treatment of the tissue with pressure waves of shock waves with the aforementioned stimulation by current, the latter, according to the invention, is generated via the applicator part itself or by a cap of the applicator part on the distal side of the applicator part and/or by a applicator-retaining part fixed to the housing and which is applied by this into the body, enhances the positive effect of the pressure waves and/or shock waves for removing cellulite or folds. This embodiment can also be used for the catabolism of fat (lipolysis).

The high frequency current preferably has a frequency in the range of about 0.5 MHz to about 50 MHz. The high frequency of about 10 to about 50 MHz has the advantage that in case that monopolar high frequency current is applied, a neutral electrode can be dispensed with, which otherwise has to be arranged on the body.

In order to avoid burnings at the skin surface, the electrode can be cooled in case of the use of high frequency current, or the electrode is permanently moved upon the application of the high frequency current.

In further preferred embodiments, the applicator part has at least one ultrasound emitter, and/or the applicator part has a cap arranged on the distal side and having at least one ultrasound emitter, and/or the at least one ultrasound emitter is arranged in or on a headpiece of the housing.

The at least one ultrasound emitter or the several ultrasound emitters preferably defines/define a circular emitter surface.

The at least one aforementioned ultrasound emitter thus generates ultrasound outside the body, and this ultrasound is applied, in addition to the mechanical pressure waves or shock waves, which have quite a low frequency, into the body, such that the low-frequency pressure waves or shock waves can superpose with the high-frequency ultrasound waves in the area to be treated.

In further preferred embodiments, the applicator part has at least one light source, and/or the applicator part has a cap arranged on the distal side and having at least one light source, and/or at least one light source is arranged in or on a headpiece of the housing, wherein the at least one light source is chosen from the group including an LED, a laser or a laser-LED.

In this case, it is also preferred if the one or more light sources define a circular light emission surface.

By combining light with the mechanical pressure waves or shock waves in the area of the body to be treated, it is also possible to achieve better treatment results than with mechanical pressure or shock waves alone. The action of the light emitted by the at least one light source likewise provides more intensive circulation of blood and stimulation of cells in the tissue of the area to be treated, as a result of which the mechanical pressure waves or shock waves, which are applied at the same time, exert an improved action. It will be appreciated that the light source can comprise a plurality of LEDs or laser-LEDs, which can be arranged, for example in a ring shape, in or on the applicator or on the cap of the applicator.

With a combined application of mechanical pressure waves or shock waves and light, it is again advantageous in the case of use of the apparatus for the treatment of cellulite or folds, if the light applied by the light source is able to heat the surface superficially or in the depth, until a shrinking of collagen fibers occurs. This combined application can also be used for the catabolism of fat (lipolysis).

Such a heating can be achieved by the applied light in that the at least one light source radiates infrared light. One or more infrared LED's or infrared laser LED's can be used as the light source for this purpose. Differently from light of shorter wavelengths, the infrared light is able to achieve such a heating of the skin or the tissue lying under the skin that collagen fibers shrink, as described above in the context of the application of high frequency current. The temperatures necessary for this effect lie in an estimated range of about 50° C. to about 80° C.

In particular, the aforementioned ultrasound emitters or aforementioned light sources are preferably arranged in or on the headpiece of the apparatus housing in the vicinity of the applicator part, which reduces the costs for maintenance of the apparatus, especially in the case of exchangeable applicator parts. The headpiece of the housing is preferably detachable and preferably serves as an applicator-retaining part for securing said applicator on the handpiece.

According to another aspect, the impacts that can act repeatedly on the proximal end of the applicator part are preferably effected by at least one percussion part that is moved along an accelerator path.

In an advantageous embodiment of this aspect, the percussion part is made from a material of low specific weight, for example plastic or a light metal, for example titanium, aluminium or the like.

The percussion part preferably has a mass of less than 10 g, preferably of less than g, more preferably of less than 3 g.

The embodiment of the at least one percussion part with low weight has the advantage that, in contrast to the known apparatus, pulse frequencies or shock frequencies of more than 20 Hz can be achieved, without this requiring an increase in the acceleration path of the percussion part. The apparatus thus remains easy to handle, and it is not necessary, in the case of a pneumatic drive of the percussion part, to increase the pressure of the pneumatic drive. A higher pulse frequency or shock frequency has the advantage that resonances can be more efficiently stimulated in the tissue to be treated, and such resonances can further improve the therapeutic effect of the pressure waves or shock waves.

It is also preferable if the percussion part has recesses, hollows, depressions or openings.

The recesses, hollows, depressions or openings also lead to a reduced weight of the percussion part, without having to use a particularly lightweight material, such as the ones mentioned above, for producing the percussion part. Thus, in the context of the present embodiment, the percussion part can also be made of a metal of higher density and greater hardness, and therefore of greater resistance to wear, for example steel, in which case the recesses, hollows, depressions or openings then substantially reduce the mass of the percussion part.

It is also preferable if the percussion part, upon impacting the applicator part, is accelerated to an impacting speed of greater than 20 m/s.

A high impact speed of the percussion part, of more than 20 m/s, which is not achieved in the known apparatus, has the advantage that, on the one hand, the pulse frequency of the generated impacts can be increased, and, on the other hand, the higher impacting speed means that, despite the lower mass of the percussion part according to the aforementioned embodiments, a sufficiently high pressure-wave energy is obtained in the applicator part and, consequently, after application, in the area of the body to be treated.

In another preferred embodiment, the percussion part has a length which is not substantially longer than twice the diameter of the percussion part.

The shortest possible configuration of the percussion part has the advantage of a lower mass of the percussion part for achieving a higher pulse frequency, and a length corresponding to about twice the diameter of the percussion part advantageously avoids tilting or jamming of the percussion part on its acceleration path.

According to another aspect of the invention, a medical apparatus for treatment of the human or animal body by mechanical pressure waves or shock waves is provided, comprising a device designed to generate the pressure waves or shock waves, the device for generating the pressure or shock waves having at least one applicator part having a proximal end and a distal end, wherein the at least one applicator part can be subjected to impacts at the proximal end in order to generate the pressure waves or shock waves and, at a distal end, apply the pressure waves or shock waves into the body, and at least two percussion parts which are each moved along a respective acceleration path and which each impacts on the proximal end of the applicator part.

The advantage of a pressure wave or shock wave generator mechanism with a plurality of percussion parts is that the individual percussion parts can each be designed with a low mass, without reducing the overall impulse on the applicator part. The several percussion parts thus provide an impulse amplification with at the same time a lower mass of the percussion part, and without increasing the acceleration path.

In this case, the percussion parts can impact substantially simultaneously on the applicator part, in order to exert as great an overall impulse as possible on the applicator part, as a result of which pressure waves or shock waves of high energy can be generated, or the percussion parts can also impact in succession on the applicator part, resulting in the advantage of a higher pulse frequency.

In the latter case, it is also preferable if the applicator part can be tilted from its longitudinal axis, and if the percussion parts impact on the applicator part outside the longitudinal axis.

In this embodiment, the percussion parts impacting in succession and outside the longitudinal axis on the applicator part, which can be tilted from its longitudinal axis, cause a tilting or wobbling movement of the applicator part, depending on whether two percussion parts or three or more percussion parts are present. The tilting or wobbling movement of the applicator part, in addition to the application of the pressure waves or shock waves, provides a massaging action on the tissue. This embodiment represents in particular an advantageous embodiment of the abovementioned aspect of the invention according to which the apparatus is additionally designed to generate at least one further action parameter outside the body and apply it into said body, which action parameter includes a mechanical action.

Further advantages and features will become evident from the following description and from the attached drawing.

It will be appreciated that the aforementioned features to be explained in more detail below can be used not only in the respectively cited combination, but also in other combinations or singly, without departing from the scope of the present invention.

### BRIEF DESCRIPTION OF THE DRAWINGS

Illustrative embodiments of the invention are depicted in the drawings and are described in more detail below with reference to said drawings, in which:

FIG. 1 shows a medical apparatus for treatment of the human or animal body by mechanical pressure waves or shock waves, in schematic longitudinal section;

FIG. 2 shows a view of the distal end of an applicator part of the apparatus from FIG. 1 according to one illustrative embodiment;

FIG. 3 shows a view of the distal end of an applicator part of the apparatus from FIG. 1 according to another illustrative embodiment;

FIG. 4 shows a view of the distal end of the apparatus from FIG. 1 according to yet another illustrative embodiment;

FIG. 5 shows another illustrative embodiment of a medical apparatus for treatment of the human or animal body by mechanical pressure waves or shock waves, in schematic longitudinal section; and

FIG. 6 shows a further illustrative embodiment of a medical apparatus for treatment of the human or animal body by mechanical pressure waves or shock waves, in schematic longitudinal section.

### DETAILED DESCRIPTION OF EXEMPLARY PREFERRED EMBODIMENTS

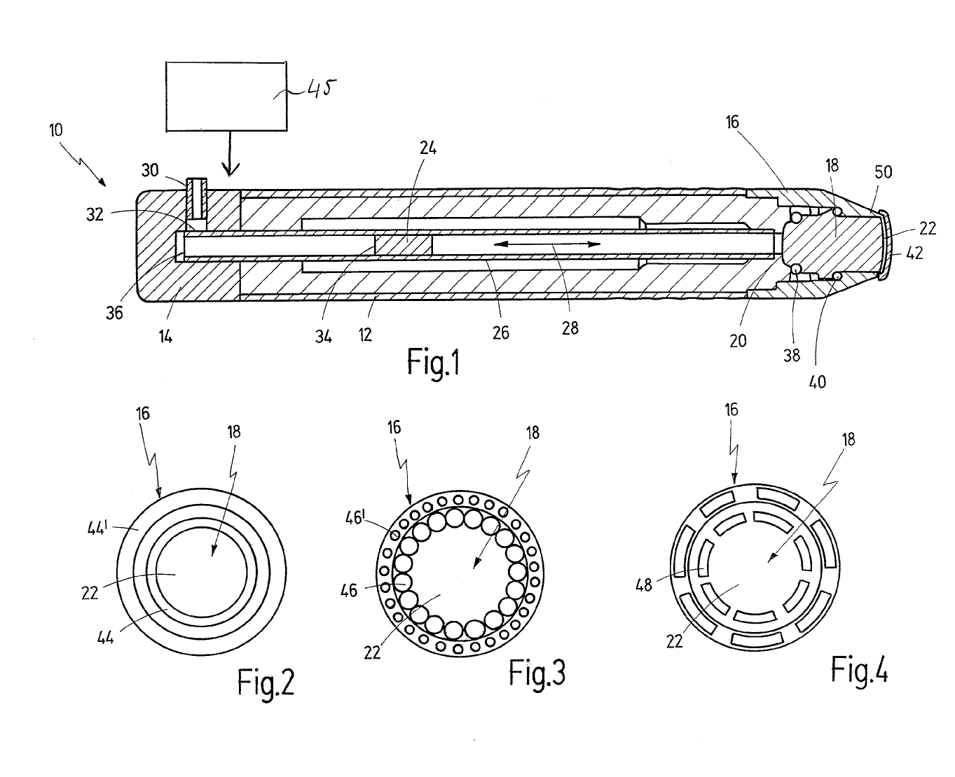

In FIG. 1, a medical apparatus for treatment of the human or animal body by mechanical pressure waves or shock waves is designated overall by reference number 10.

The apparatus 10 is used for the treatment of pain, for example, or generally for soft-tissue treatment.

The apparatus 10 generally has a housing 12 with a proximal endpiece 14 and a distal headpiece 16. The apparatus 10 is designed in the form of a rod-shaped hand-piece.

The apparatus 10 has a device which is designed to generate pressure waves or shock waves and which has an applicator part 18 arranged in the distal headpiece 16 at the distal end of the housing 12. The headpiece 16 serves as an applicator holder for retaining the applicator part 18 on the handpiece.

The applicator part 18 has a proximal input surface 20 and a distal output surface 22 for the pressure waves or shock waves. The pressure waves or shock waves are generated in the applicator part 18 via the input surface 20. For this purpose, the housing 12 accommodates a percussion part 24, which can be moved to and from according to the double arrow 28 in a guide tube 26 extending in the longitudinal direction of the housing 12, in order to repeatedly exert an impact on the input surface 20 of the applicator part 18. The percussion part 24 is moved in the distal direction by means of compressed air, for which purpose the proximal endpiece 14 of the housing 12 is provided with a compressed-air inlet 30 through which compressed air from an external compressed-air source (not shown) is introduced in pulses into the guide tube 26 through an opening 32 thereof; the introduced compressed air acts on a proximal surface 34 of the percussion part 24 and, as a result, the percussion part 24 is accelerated from its starting position at 36 towards the applicator part 18.

Each time the percussion part 24 strikes the input surface 20 of the applicator part 18, a pressure wave or shock wave is induced in the latter and is propagated through the applicator part 18 to the output surface 22 and leaves the latter in order to be applied into the body of a patient.

Upon each impact by the percussion part 24, the applicator part 18 executes only a minimal stroke, the applicator part 18 being clamped elastically between a proximal elastomeric O-ring 38 and a distal elastomeric O-ring 40. However, the applicator part 18 can also be mounted in such a way that it can execute a comparatively long stroke of up to 5 mm.

The apparatus 10 is not only designed to generate and apply mechanical pressure waves or shock waves; it is additionally designed to generate outside the body at least one other physiological action parameter and then apply the latter into the human or animal body, said action parameter including an electric current, electromagnetic waves, in particular light, ultrasound and/or a further mechanical action.

The at least one other action parameter is preferably generated by the applicator part 18 or by a cap 42 arranged on the distal output surface 22 and/or by the headpiece 16 of the housing, particularly in a distal area thereof, and applied into the body of a patient.

FIG. 2 shows an embodiment of the applicator part 18 in which the applicator part 18 has, on its output surface 22, an electrode 44 that can be supplied with current. As an alternative to this, however, the aforementioned cap 42 can also have the electrode 44.

The electrode 44 is in particular configured as a circular ring or disc and, in the illustrative embodiment shown, it has the shape of a circular ring. It is also possible for the entire applicator part 18 itself, or part thereof, to be designed as an electrode. As is also shown in FIG. 2, the headpiece 16 of the housing can additionally or alternatively have an electrode 44′ or can itself be designed as an electrode.

In addition to the application of the mechanical pressure waves or shock waves, the electrode 44 and/or 44′ can be used to apply a current, in particular a therapeutic alternating current, into the area of the body to be treated, such that the actions of the mechanical pressure waves or shock waves and of the current superpose one another in the area being treated.

Instead of a therapeutic alternating current, a monopolar or bipolar high frequency current can be applied into the body into the treatment area via the electrode 44 and/or 44′, which high frequency current is able to heat the skin superficially or in the depth to such an extent that collagen fibers shrink. This effect is particularly advantageous for the treatment of cellulite or folds. A power supply source 25, which provides bipolar or monopolar high frequency current, accordingly, is schematically shown in FIG. 1. The high frequency voltage is applied to the electrode 44 and/or 44′ accordingly. In case of the application of bipolar high frequency current, one of the two electrodes 44, 44′ can form the neutral electrode, and the other of the two electrodes the active electrode. The current flow then occurs between the two electrodes with a low depth of penetration into the skin.

In case of the application of monopolar high frequency current, one or both the electrodes 44, 44′ can form the active electrodes, respectively, while a neutral electrode (not shown) is applied to the body of the patient, usually remote from the treatment location. Such an additional neutral electrode, however, can be dispensed with, if the high frequency current is applied with a high frequency, for example, in the range of about 10 MHz to about 50 MHz. The frequency of the high frequency current, however, generally can also be lower, for example, in the range of about 0.5 to 4 MHz.

FIG. 3 shows an illustrative embodiment of the applicator part 18 in which the applicator part 18 has a plurality of light sources 46 which, in addition to the mechanical pressure waves or shock waves, apply light, in particular therapeutic light, into the area of the patient's body to be treated. Alternatively or in addition, light sources 46′ can be arranged in or on the distal area 50 of the headpiece of the housing.

In the illustrative embodiment shown, the light sources 46 or 46′ consist of an array of light-emitting diodes (LEDs), although they can also comprise a laser or laser LEDs.

If the apparatus 10 is to be used for the treatment of cellulite or folds, the light sources 46 or 46′ preferably are light sources which radiate infrared light. The infrared light also causes a heating of tissue like the above-mentioned high frequency current, which heating of tissue has the effect that skin fibers or collagen fibers are caused to shrink, i.e. it has the effect to produce a denaturation of these fibers at least in part. In this case, infrared LED's or infrared laser LED's can be used as light sources 46 or 46′.

The light sources 46 or 46′ are arranged in a circle shape. It is also possible, once again, for the light source or light sources 46 to be provided on the cap 42.

FIG. 4 shows an illustrative embodiment of the applicator part 18 in which a plurality of ultrasound emitters 48 are arranged on the output surface 22. The ultrasound emitters 48 generate ultrasound which, in addition to the mechanical pressure waves or shock waves, is then applied into the patient's body from the ultrasound emitters 48 or the applicator part 18.

In the illustrative embodiment shown, the ultrasound emitters 48 are arranged in a circle shape on the output surface 22 of the applicator part 18 and define a circular ultrasound emission surface. The ultrasound emitters 48 can also be arranged on the cap 42, or the headpiece 16 of the housing can have ultrasound emitters 48′ in or on the distal area 50.

It will be appreciated that the illustrative embodiments according to FIGS. 2 to 4 can also be combined with one another, i.e. the applicator part 18, the applicator cap 42 and/or the headpiece 16 of the housing can have combinations of the electrode 44, the light sources 46 and/or the ultrasound emitters 48.

In the cases where the electrode(s), light source(s), ultrasound emitters are provided in or on the applicator part 18, the energy supply to these elements can be inductive or capacitive via sliding contacts.

Referring to FIG. 1 again, the percussion part 24 is made from a material of low specific weight. Such a material can in particular be plastic or a light metal, for example titanium, aluminium or the like. By using a material of low specific weight, the percussion part 24 has a low mass, such that, without increasing the acceleration path in the guide tube 26, the percussion part 24 impacts the input surface 20 of the applicator part 18 at a high speed of more than 20 m/s.

The mass of the percussion part 24 is preferably less than 10 g, preferably less than 5 g, more preferably less than 3 g.

The mass of the percussion part 24 can be further reduced by providing the percussion part 24 with recesses, hollows, depressions or openings. In the simplest case, for example, the percussion part 24 can have a continuous or blind longitudinal bore, such that the percussion part 24 is designed overall or partially in the form of a sleeve. The openings can also be formed in the percussion part 24 in a direction transverse to the longitudinal direction of the percussion part 24.

To ensure that the percussion part 24 does not become canted in the guide tube 26, its length in the direction of the double arrow 28 is greater than or equal to twice its diameter in the direction transverse to the double arrow 28, the length of the percussion part 24 preferably being more or less equal to twice its diameter.

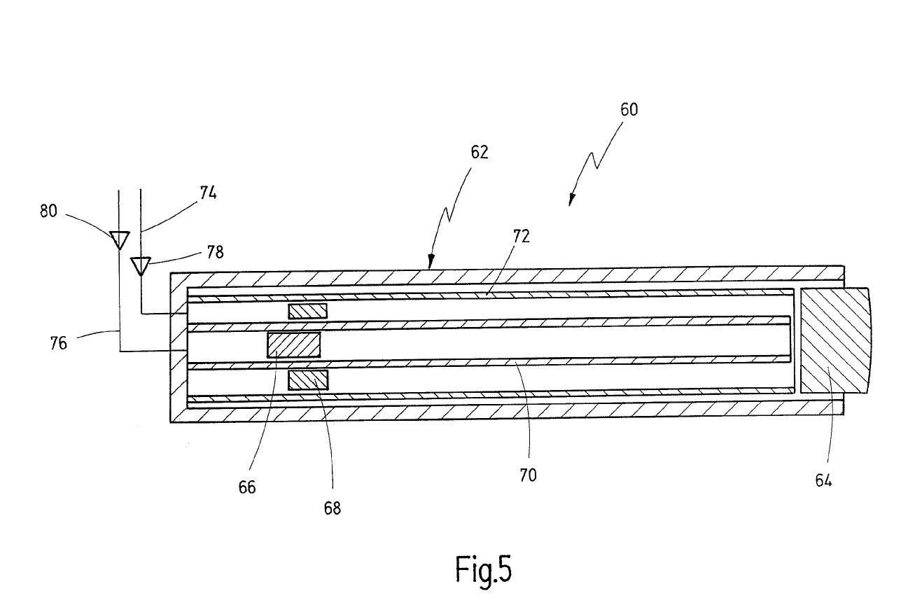

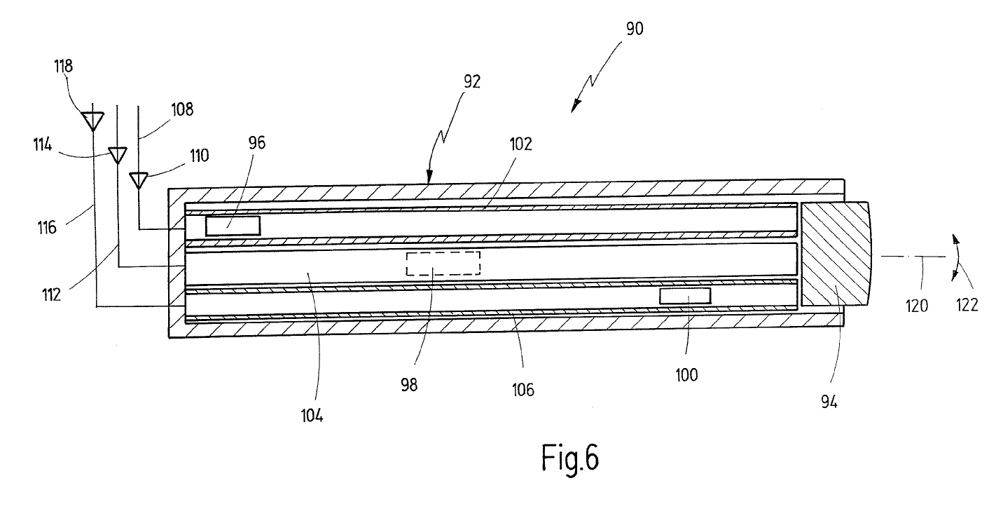

Further illustrative embodiments of a medical apparatus for treatment of the human or animal body by mechanical pressure waves or shock waves will be described with reference to FIGS. 5 and 6.

FIG. 5 shows a medical apparatus 60 with a housing 62 and an applicator part 64 for generating and applying pressure waves or shock waves into the human or animal body. The applicator part 64 can have one or more of the configurations according to FIGS. 2 to 4, in which respect reference is made to the above description. In contrast to the apparatus 10, the apparatus 60 has a device for generating pressure waves or shock waves which comprises, in addition to the applicator part 64, two percussion parts 66 and 68. The percussion part 66 corresponds to the percussion part 24 in FIG. 1, with the percussion part 66 being moved to and from in a guide tube 70 that corresponds to the guide tube 26 in FIG. 1.

In the illustrative embodiment shown in FIG. 5, the percussion part 68 is designed as an annular sleeve which is arranged concentrically around the percussion part 66 and is moved to and from in a guide tube 72 that concentrically surrounds the guide tube 70 and is designed as a cylindrical tube.

In the illustrative embodiment in FIG. 5, the percussion parts 66 and 68 are connected to compressed-air conduits 74 and 76 with corresponding control valves 78 and 80 and are subjected to pressure in such a way that the percussion parts 66 and 68 impact the applicator part 64 simultaneously. To ensure that the percussion parts 66 and 68 are accelerated synchronously, their masses are adapted to each other, or the pressure of the air or gas acting in pulses on the proximal end of the percussion parts 66 and 68 is correspondingly adapted to the two percussion parts 66 and 68.

The presence of at least two percussion parts 66 and 68 that impact the applicator part 64 simultaneously means that a correspondingly greater energy of the pressure waves or shock waves is generated in the applicator part 64 and applied into the patient's body.

FIG. 6 shows a medical apparatus 90 for treatment of the human or animal body by mechanical pressure waves or shock waves that comprises a housing 92 and, at the distal end, an applicator part 94.

The apparatus 90 has a device which generates pressure waves or shock waves and which has three or four percussion parts 96, 98 and 100.

The percussion part 96 can be moved to and from in a guide tube 102, the percussion part 98 in a guide tube 104, and the percussion part 100 in a guide tube 106. The guide tube 102 is subjected to pressure via a compressed-air conduit 108 with control valve 110, the guide tube 104 via a compressed-air conduit 112 with control valve 114, and the guide tube 106 via a compressed-air conduit 116 with control valve 118, so as to accelerate the respective percussion part 96, 98 and 100 from a proximal starting position to the applicator part 94.

In contrast to the illustrative embodiment in FIG. 5, the percussion parts 96, 98 and 100 in the apparatus 90 impact in succession on the applicator part 94. In this way, it is possible to achieve a greater percussion frequency or pulse frequency in the generation of the mechanical pressure waves or shock waves, in particular a percussion frequency or pulse frequency of more than 30 Hz or even of more than 100 Hz.

In this illustrative embodiment, the applicator part 94 can be mounted in the housing 92 in such a way that it is able to tilt relative to a longitudinal centre axis 120 of the applicator part 94, as is illustrated by a double arrow 122. In this case, the percussion parts 96, 98, 100 and guide tubes 102, 104, 106 are arranged eccentrically relative to the longitudinal centre axis 120, such that the percussion parts 96, 98, 100 impact on the applicator part 94 outside the longitudinal centre axis 120 and cause a corresponding tilting of the applicator part 94 relative to the longitudinal centre axis 120. As a result of the percussion parts 96, 98 and 100 impacting in periodic succession on the applicator part 94, the latter makes a kind of wobbling movement about the longitudinal centre axis 120. This wobbling movement represents a further mechanical action that can be applied into the patient's body in addition to the mechanical pressure waves or shock waves also generated in the applicator part 94 by the percussion parts 96, 98 and 100, as a result of which an additional massaging action is generated by the applicator part 94.

In the illustrative embodiment shown in FIG. 6, the percussion parts 96, 98, 100 and the guide tubes 102, 104, 106 can be arranged about the longitudinal centre axis 120 at an angle of 120° from one another. It is also possible to provide four or more percussion parts, which are accordingly arranged about the longitudinal centre axis 120 at angles of 90° to one another or at another suitable angle to one another, such that they each impact eccentrically on the applicator part 94.

The control valves 110, 114, 118 in the compressed-air conduits 108, 112, 116 are suitably clocked to allow the percussion parts 96, 98, 100 to impact individually and in succession on the applicator part 94.

Moreover, the applicator part 94 can additionally be configured according to one or more of the embodiments in FIGS. 2 to 4.

What is claimed is:

1. A medical apparatus for treatment of the human or animal body by mechanical pressure waves or shock waves, comprising
a device designed to generate the pressure waves or shock waves, said device having
at least one applicator part having a proximal end and a distal end, and which can be subjected to impacts at said proximal end in order to generate said pressure waves or shock waves and which applies said pressure waves or shock waves into said body at said distal end,
wherein said apparatus is additionally designed to generate outside said body, and to apply into said body, at least one further physiological action parameter chosen from the group consisting of electrical current, electro-magnetic waves, ultrasound, a further mechanical action beyond said pressure waves or shock waves.

2. The apparatus of claim 1, further comprising at least one electrode that can be supplied with current.

3. The apparatus of claim 2, further comprising a power apply source which supplies the at least one electrode with a high frequency current.

4. The apparatus of claim 3, wherein said high frequency current is a bipolar high frequency current.

5. The apparatus of claim 3, wherein said high frequency current is a monopolar high frequency current.

6. The apparatus of claim 3, wherein said high frequency current has a frequency in the range of about 0.5 MHz to about 50 MHz.

7. The apparatus of claim 2, wherein one of the applicator part, a cap arranged on a distal side of said applicator part, a headpiece of the housing has said at least one electrode.

8. The apparatus of claim 2, wherein said at least one electrode is circular.

9. The apparatus of claim 1, further comprising at least one ultrasound emitter.

10. The apparatus of claim 9, wherein one of said applicator part, a cap arranged on a distal side of said applicator part, a headpiece of the housing has said at least one ultrasound emitter.

11. The apparatus of claim 9, wherein said at least one ultrasound emitter defines a circular emitter surface.

12. The apparatus of claim 1, further comprising at least one light source chosen from the group consisting of an LED, a laser, a laser-LED.

13. The apparatus of claim 1, further comprising at least one light source, wherein said at least one light source is an infrared light radiating light source.

14. The apparatus of claim 12, wherein one of said applicator part, a cap arranged on a distal side of said applicator part, a headpiece of a housing has said at least one light source.

15. The apparatus of claim 12, wherein said at least one light source defines a circular light emission surface.

16. The apparatus of claim 1, wherein said device for generating said pressure waves or shock waves further has at least one percussion part that is moved along an acceleration path and which effects said impacts that can act repeatedly on said proximal end of said applicator part.

17. The apparatus of claim 16, wherein said at least one percussion part is made from a material of low specific weight.

18. The apparatus of claim 17, wherein said percussion part is made from a material chosen from the group consisting of plastic, light metal, titanium, aluminium.

19. The apparatus of claim 16, wherein said percussion part has a mass of less then 10 g.

20. The apparatus of claim 16, wherein said at least one percussion part has recesses, hollows, depressions or openings.

21. The apparatus of claim 16, wherein said at least one percussion part is accelerated to an impacting speed of greater than 20 m/s upon impacting said applicator part.

22. The apparatus of claim 16, wherein said at least one percussion part has a length which is not substantially longer than twice a diameter of said at least one percussion part.

23. The apparatus of claim 16, wherein said device for generating said pressure waves or shock waves has at least two percussion parts each of which are moved along a respective acceleration path, said at least two percussion parts repeatedly impact on said proximal end of said applicator part.

24. The apparatus of claim 23, wherein said at least two percussion parts impact substantially simultaneously on said applicator part.

25. The apparatus of claim 23, wherein said at least two percussion parts impact in succession on said applicator part.

26. The apparatus of claim 25, wherein said applicator part can be tilted from its longitudinal center axis, and wherein said at least two percussion parts impact on said applicator part outside a longitudinal center axis of said applicator part.

27. An apparatus for treatment of the human or animal body by mechanical pressure waves or shock waves, comprising
a device designed to generate said pressure waves or shock waves, said device for generating said pressure or shock waves having
at least one applicator part having a proximal end and a distal end, wherein said at least one applicator part can be subjected to impacts at said proximal end in order to generate said pressure waves or shock waves and, at a distal end, apply said pressure waves or shock waves into said body, and
at least two percussion parts which are each moved along a respective acceleration path and which each impacts on said proximal end of said applicator part.

28. The apparatus of claim 27, wherein said at least two percussion parts impact substantially simultaneously on said applicator part.

29. The apparatus of claim 27, wherein said at least two percussion parts impact in succession on said applicator part.

30. The apparatus of claim 27, wherein said applicator part can be tilted from its longitudinal center axis, and wherein said at least two percussion parts impact on said applicator part outside a longitudinal center axis of said applicator part.
